(19) United States
(12) Patent Application Publication
(10) Pub. No.: US 2026/0069804 A1
(43) Pub. Date: Mar. 12, 2026

(54) SYSTEMS AND METHODS FOR ASSURED AUTONOMOUS MECHANICAL VENTILATION

(71) Applicant: THE JOHNS HOPKINS UNIVERSITY, Baltimore, MD (US)

(72) Inventors: Khalid HALBA, North Potomac, MD (US); Russell H. TAYLOR, Severna Park, MD (US); Jules BERGMANN, Baltimore, MD (US); Kimia GHOBADI, Baltimore, MD (US); Anton T. DAHBURA, Hagerstown, MD (US); James FACKLER, Baltimore, MD (US); Antwan D. CLARK, Pikesville, MD (US)

(21) Appl. No.: 19/107,866

(22) PCT Filed: Aug. 31, 2023
(86) PCT No.: PCT/US2023/031588

§ 371 Date: Feb. 28, 2025

Related U.S. Application Data
(60) Provisional application No. 63/374,269, filed on Sep. 1, 2022.

(51) Int. Cl. A61M 16/00

(52) U.S. Cl. CPC A61M 16/026; A61M 16/0051; A61M 2205/15; A61M 2205/502; A61M 2205/52; A61M 2230/20; A61M 2230/42

(57) ABSTRACT

A system for generating an assurance value for autonomous ventilation of a patient is provided, and includes a computer, a storage device, a ventilator, and a device for acquiring patient physiological data associated with the patient. Instructions are provided that, when executed by the computer, cause the computer to generate the assurance value using a method which includes: determining an adaptive patient digital twin and a correlated ventilator model; receiving patient physiological data during a time period; processing the adaptive patient digital twin and the correlated ventilator model to generate expected patient physiological data during a second time period; receiving the patient physiological data during the second time period; and processing the patient physiological data during the second time period and the expected patient physiological data during the second time period to generate an assurance value associated with the adaptive patient digital twin and the correlated ventilator model.

### CROSS-REFERENCE TO RELATED APPLICATIONS

This application is the national stage entry of International Patent Application No. PCT/US2023/031588, filed on Aug. 31, 2023, and published as WO 2024/049934 A1 on Mar. 7, 2024, which claims the benefit of U.S. Provisional Application No. 63/374,269, filed on Sep. 1, 2022, which are hereby incorporated by reference in their entireties.

### TECHNICAL FIELD

This disclosure relates to systems and methods for autonomous mechanical ventilation.

### BACKGROUND

Mechanical ventilation is both life-saving and life-threatening. Possibly the most important pulmonary critical care insight in the last few decades is recognition that “lung protective” mechanical ventilation strategies that tolerate mild acidemia and hypoxemia improve outcomes, and in randomized clinical trials, save lives.

Despite internationally accepted guidelines for all phases of mechanical ventilation therapy, guideline adherence is abysmal. Today, when a clinician decides to adjust a ventilator, they must go to the bedside (often a risk-laden space). Further, physiologic and ventilator data have limited availability and persistence. Consequently, ventilator changes typically occur at arbitrary fixed intervals or if the patient's state demands immediate (crisis) attention. Friction to information flow and the decision-making bottleneck make it difficult to find patient states that precede these crisis-necessitated interventions, which if discovered and adjusted would mitigate the crises. Conversely, the friction to information flow and the decision-making bottleneck encourages conservative settings to hedge against potential decompensation that slows de-escalation as patients improve which unnecessarily prolongs the patient's exposure to mechanical ventilation (and its attendant complications).

This state of practice causes patients to be ventilated longer than necessary. Ventilator settings are often mismatched to patient requirements, increasing risk of iatrogenic injury and subsequent morbidity and mortality.

Autonomous Mechanical Ventilation (also known as “Closed-loop Controlled Mechanical Ventilation”) is an improvement on conventional mechanical ventilation. Systems under closed-loop control, in general, are designed to generate a control action for a “process variable” (PV) of the system based on the value of the PV. Often, the PV for a system under closed-loop control is a physical variable of a device in the system. As described in the article “The dawn of physiological closed-loop ventilation-a review” by P. von Platen et al. (Crit Care 24, 121 (2020)), a PV for a closed-loop control medical system that includes a patient can include a physiological variable of the patient.

FIG. 1 illustrates an exemplary prior art medical system 100 under closed-loop control. In FIG. 1, mechanical ventilator 120, under control (125) of controller 110, provides physiological assistance (135) to patient 130, where certain physiological variables of patient 130 are used as feedback (145), and compared with reference values (107) provided by clinician 105. The error (115) from this comparison 140 is used by controller 110 to further refine its control of ventilator 120 so as to minimize error 115.

Other articles discussing closed-loop mechanical ventilation include: “Automation of Mechanical Ventilation” by R. D. Branson (Critical Care Clinics. 2018; 34(3): 383-94); “Principles and history of closed-loop controlled ventilation” by J. X. Brunner (Respir Care Clin N Am. 2001 September; 7 (3): 341-62, vii); “PEFIOS: an expert closed-loop oxygenation algorithm” by D. Waisel, et al. (Medinfo. 1995; 8 Pt 2:1132-1136); and “Please Welcome the New Team Member: The Algorithm*” by J. C. Fackler et al. (Pediatric Critical Care Medicine. 2019 December; 20 (12): 1200-1).

While mechanical ventilators under closed-loop control are an improvement over conventional mechanical ventilators, there remains a need to adjust and monitor the ventilator in real-time—not simply at the clinician's convenience—and to deliver the support currently required by the patient, as opposed to excess or inadequate support based on out-of-date clinician assessments of patient state.

### SUMMARY OF THE INVENTION

Disclosed herein are systems and methods for mechanical ventilation that are autonomous and assured. As used herein, “autonomous” means being able to make correct decisions based on the patient's physiologic state without a human clinician in the loop. Further still, as used herein, “assured” means capable of recognizing situations outside a system's capability to manage, so that human intervention is required.

According to an exemplary embodiment of the present disclosure, a system for generating an assurance value for autonomous ventilation of a patient includes at least one computer, at least one storage device, a ventilator coupled to the computer, where the ventilator is associated with the patient, and at least one device for acquiring patient physiological data associated with the patient, the computer being coupled to the device for receiving acquired patient physiological data. In an embodiment, the storage device stores instructions that, when executed by the computer, cause the computer to perform a method of generating the assurance value. In an embodiment, the method of generating the assurance value includes: determining at least one adaptive patient “digital twin” (i.e., a computer simulation model reflecting relevant physiological and other relevant data associated with a patient) and a correlated ventilator model; receiving the acquired patient physiological data during a first time period; processing the adaptive patient digital twin and the correlated ventilator model to generate expected patient physiological data during a second time period after the first time period; receiving the acquired patient physiological data during the second time period; and processing the acquired patient physiological data during the second time period and the expected patient physiological data during the second time period to generate an assurance value associated with the adaptive patient digital twin and the correlated ventilator model.

According to another exemplary embodiment of the disclosure, a system for generating an assurance value includes the system of the previous embodiment, where the ventilator is configured to provide first ventilator data corresponding to said first time period to said computer, and where the step of processing the at least one adaptive patient digital twin and the correlated ventilator model further includes processing the first ventilator data to generate expected ventilator data during the second time period. Further still, in an embodiment, the step of receiving the acquired patient physiological data during the second time period includes receiving second ventilator data corresponding to said second time period, and the step of processing the acquired patient physiological data during the second time period and the expected patient physiological data during the second time period includes processing the second ventilator data received during the second time period and the expected ventilator data during the second time period.

According to further embodiments, a system for generating an assurance value consistent with this disclosure includes any of the previous embodiments where the assurance value is generated based upon a threshold. Further still, a system for generating an assurance value consistent with this disclosure includes any of the previous embodiments where divergence between the acquired patient physiological data during the second time period and the expected patient physiological data during the second time period is used to generate a modification of the at least one adaptive patient digital twin and correlated ventilator model.

Moreover, consistent with the current disclosure, a system for generating an assurance value includes any of the previous embodiments, where the method of generating the assurance value includes generating a model for patient prognosis during a third time period that provides an indication of patient prognosis at least a fixed time amount past the third time period. In embodiments, the fixed time amount can be 1 hour, 2 hours, 3 hours, . . . 24 hours, etc. Further still, a system for generating an assurance value consistent with this disclosure includes any of the previous embodiments where the method of generating the assurance value further includes providing an alert when the indication of patient prognosis at least the fixed time amount past the third time period is below a set value.

Advantages of the embodiments will be set forth in part in the description which follows, and in part will be understood from the description, or may be learned by practice of the invention. The advantages will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

FIG. 1 illustrates an exemplary control loop for a closed-loop control mechanical ventilator system of the prior art.

FIG. 2 depicts a high-level diagram of an assured autonomous mechanical ventilation system consistent with the current disclosure.

FIG. 3 depicts an intermediate-level diagram of an assured autonomous mechanical ventilation system consistent with the current disclosure.

FIG. 4 illustrates an exemplary control loop architecture consistent with an embodiment of the present disclosure.

FIG. 5 is a high-level block diagram depicting a reinforcement-learning approach consistent with an embodiment.

FIGS. 6-7 are exemplary Markov Decision Process transition graphs for use with an aspect of the reinforcement-learning approach of FIG. 5

FIG. 8 is a high-level diagram illustrating an Artificial Intelligence Gym environment for use with aspects of the reinforcement-learning approach of FIG. 5.

FIG. 9 is a high-level diagram illustrating a Deep Q Network formulation for use with aspects of the reinforcement-learning approach of FIG. 5.

FIGS. 10-13 depict results associated with aspects of the reinforcement-learning approach of FIG. 5.

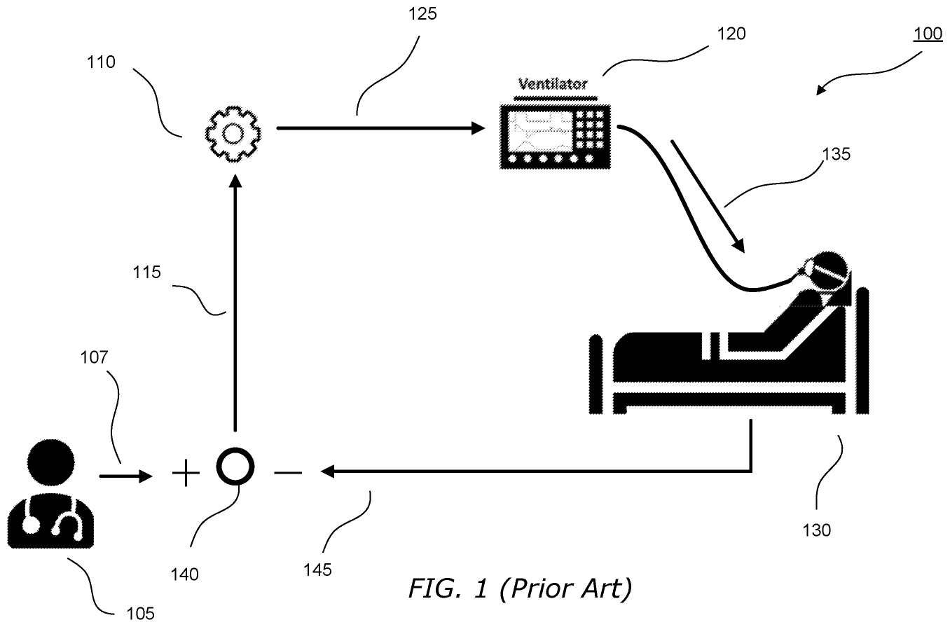

### DETAILED DESCRIPTION

Exemplary embodiments are described with reference to the accompanying drawings. In the figures, which are not necessarily drawn to scale, the left-most digit(s) of a reference number identifies the figure in which the reference number first appears. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items. It should also be noted that as used herein and in the appended claims, the singular forms “a,” “an,” and “the” include plural references unless the context clearly dictates otherwise.

Embodiments of the present disclosure relate generally to systems and methods for assured autonomous mechanical ventilation.

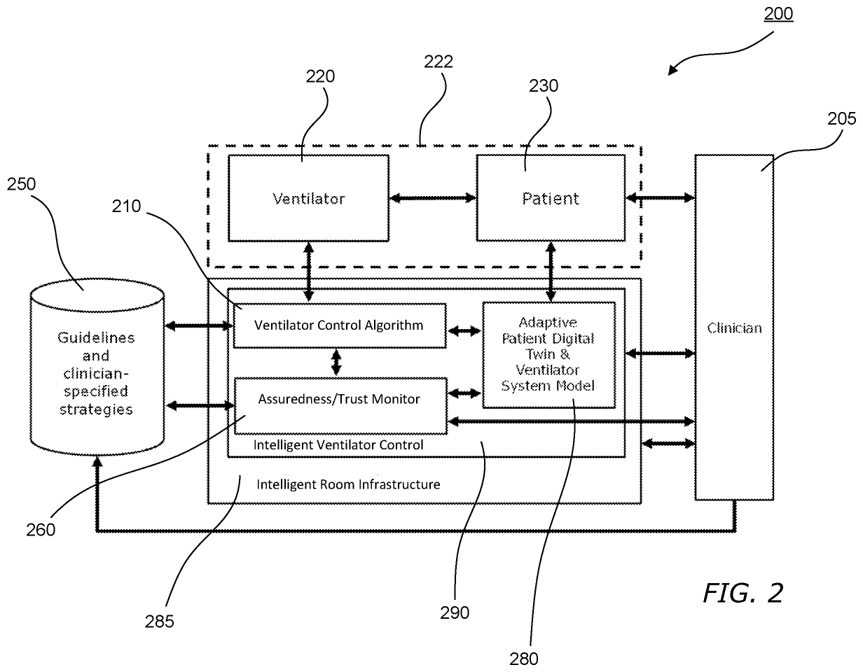

FIG. 2 depicts a high-level diagram of an assured autonomous mechanical ventilation system 200 consistent with the present disclosure. As depicted in FIG. 2, components of assured autonomous mechanical ventilation system 200 can include: ventilator-patient dyad 222, intelligent ventilator control 290, intelligent room infrastructure 285, and guidelines and clinician-specified strategies 250. The ventilator-patient dyad 222 includes ventilator 220 and patient 230. As part of the intelligent ventilator control 290, an adaptive patient digital twin and ventilator system model 280 is selected to simulate the ventilator-patient dyad 222. Further still, controller 210 (which is part of intelligent ventilator control 290) provides direct control of ventilator 220 and the simulated ventilator in the adaptive patient digital twin and ventilator system model 280. Assuredness/Trust monitor 260 is also part of intelligent ventilator control 290 and generates an assuredness value associated with system 200 through, among other things, a comparison of simulated physiological variables of the simulated patient in adaptive patient digital twin and ventilator system model 280 and actual physiological variables acquired from patient 230. Clinician 205 can monitor patient 230, the intelligent ventilator control 290, and Assuredness/Trust monitor 260, as well as the intelligent room infrastructure 285 (which can include other available environmental sensors and controls, such as those associated with a smart medical bed). Clinician 205 can also monitor and provide further updates to guidelines and clinician specified strategies 250. Further still, the guidelines and clinician specified strategies 250 can be updated by Assuredness/Trust monitor 260 and provide input to controller 210. Also available to clinician 205, or others, (not shown in FIG. 2) is a user interface that can display, or otherwise indicate, the current state of the system as well as a current target of the system. Such a display can also provide an indication of patient prognosis for a time period at least a fixed time amount in the future. In embodiments, the fixed time amount can be 1 hour, 2 hours, 3 hours, . . . 24 hours, etc. This is described further below.

Generally, a system consistent with the current disclosure can provide for assured autonomous control of a medical process (not limited to control of mechanical ventilators). Such a system, in general, can include: (a) a medical device to be autonomously controlled; (b) a control algorithm; (c) a target; (d) a “digital twin” to allow understanding; (e) a comparison of current state to current target; (f) a user interface to display the current state and current target; (g) a system to alert the human when there are potentially clinically relevant differences between current state and current target; (h) an effector; (i) a multimodal monitor to inform the current state and current state; and (j) an emergency mode. Examples of other embodiments within this framework can be tissue oxygen delivery management with control of fluid and medication infusion pumps based on a patient-based target and a digital twin of the cardio-respiratory systems with comparisons of current states and targets Another example is management of sleep state with control of medication dosing based on a multiplicity of sensors (e.g., motion and electroencephalogram).

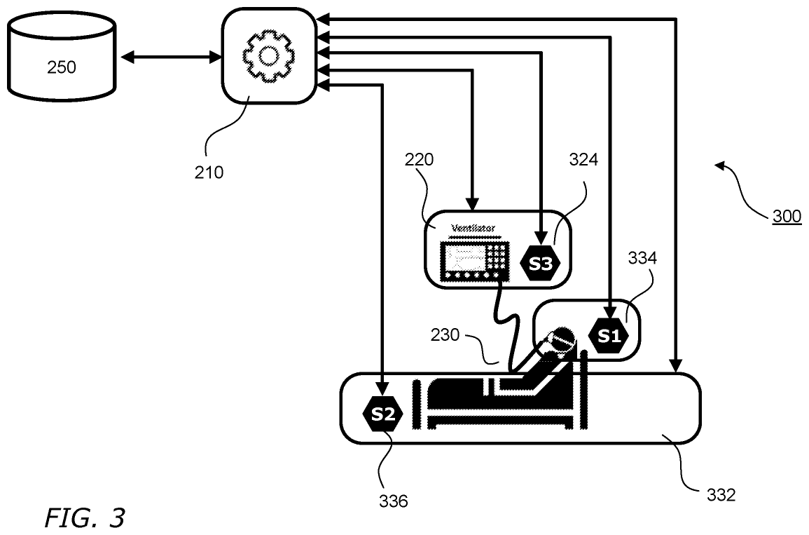

FIG. 3 depicts an intermediate-level diagram of an assured autonomous mechanical ventilation system 300 consistent with the current disclosure. Various sensors consistent with the current disclosure, available to provide feedback information to controller 210, are highlighted. As with FIG. 2, components of assured autonomous mechanical ventilation system 300 can include controller 210, which provides direct control of ventilator 220, and guidelines and clinician specified strategies 250, which is available to provide input to controller 210. Ventilator sensor 324 can provide data such as physiological data associated with patient 230, as well as operational data associated with ventilator 220 (for example, among other things, preventive maintenance data associated with ventilator 220). Sensor 334 is available to provide data associated with patient 230, such as vital signs and laboratory data. Moreover, sensor 336 (such as a sensor associated with a smart medical bed) is available to provide environmental data associated with the environment of the patient 230 and ventilator 220 dyad (for example, light, noise, and motion data).

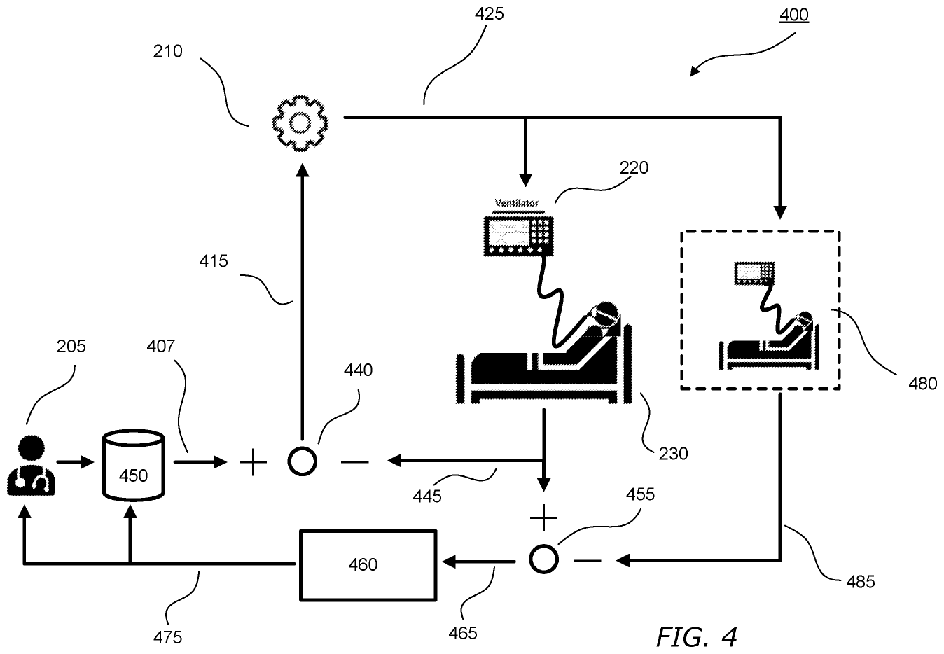

A diagram 400 depicting a system consistent with the current disclosure under two-level control is depicted in FIG. 4. As previously shown, controller 210 provides direct control of ventilator 220 through instructions 425. Instructions 425, however, are also provided to adaptive patient digital twin and ventilator system model 480. Feedback 485 from the adaptive patient digital twin and ventilator system model 480 is compared with feedback 445 from the patient 230 and ventilator 220 dyad at comparison node 455. Any difference 465 generated at comparison node 455 is fed to Assuredness/Trust monitor 460. Assuredness/Trust monitor 460 generates an assuredness value (475) which can be provided to clinician 205 and also to guidelines and clinician-specified strategies 450. The provision of the assuredness value 475 to clinician 205 and to guidelines and clinician-specified strategies 450 can be used to further update target guidelines 407. As part of a two-level control system, the updated target guidelines 407 can also be compared with the feedback 445 from the patient 230 and ventilator 220 dyad at comparison node 440 in order to generate updated instructions 415 for controller 210.

By way of example only, guidelines and clinician specified strategies 450 can include a target patient strategy for at least a fixed time amount in the future, where the fixed time amount can be 1 hour, 2 hours, 3 hours, . . . 24 hours, etc. As described above, Assuredness/Trust monitor 460 can generate an assuredness value (475) which can be provided to clinician 205 and also to guidelines and clinician-specified strategies 450, and the provision of the assuredness value 475 to clinician 205 and to guidelines and clinician-specified strategies 450 can be used to further update target guidelines 407. Where guidelines and clinician specified strategies 450 includes a target patient strategy for at least a fixed time amount in the future (for example, 12 hours in the future), then one of ordinary skill in the art would appreciate that updated target guidelines 407 at that fixed time in the future can be used to determine whether the target patient strategy is being met at that fixed time. Feedback 445 and updated instructions 415 can be used to determine the state of a patient. Among other things, assuredness value 475 (which is a function of difference 465) at that fixed time in the future can indicate no need for external intervention. Further, where the updated instructions 415 (which is based on the target guidelines 407 and the feedback 445) are consistent with the target patient strategy at the fixed time in the future, then the system can indicate that the target patient strategy at that fixed time in the future is being met. Conversely, where the updated instructions 415 (which is based on the target guidelines 407 and the feedback 445) are not consistent with the target patient strategy at the fixed time in the future, then the system can indicate that the target patient strategy at that fixed time in the future is not being met.

Accordingly, FIG. 4 depicts a two-level control system that sets support targets in line with long-term goals (the “expert”) and a model-based controller that adjusts ventilator settings to meet these targets. A predictive analytic system fuses patient measurements with model state to assess risk of decompensation. Finally, model state feeds a trust monitor that detects the need for external intervention, including situations where model predictions diverge from reality suggestive of events outside the system's scope.

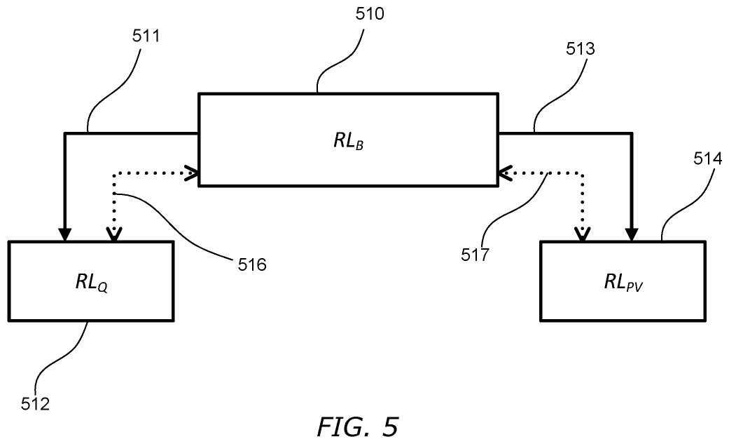

FIG. 5 is a high-level block diagram depicting a reinforcement-learning approach consistent with an embodiment. The agents depicted in FIG. 5, as described further below, can manage the controller 210 and provide the instructions 425 consistent with an embodiment. In FIG. 5, RL_{B} agent 510 governs [symbol] agent 512 and RL_{PV} agent 514 (indicated by solid lines 511 and 513). Dashed lines 516 and 517 depict communication between RL_{B} agent 510, [symbol] agent 512, and RL_{PV} agent 514. In an embodiment, RIB agent 510 is a rules-based agent that interacts with [symbol] agent 512 and RL_{PV} agent 514 and governs them to adjust the respective targets. The decision rules associated with RIB agent 510 can be designed to deliver targeted values (or intermediate targeted values) in terms of respiratory rate (RR), total cycle time (TCT), peak inspiratory pressure (PIP), positive end expiratory pressure (PEEP), and the inspiratory pressure (IP).

Further still, in an embodiment, [symbol] agent 512 can govern flow output [symbol] (t) of ventilator 220 by controlling input parameters to ventilator 220. For example, [symbol] agent 512 can dynamically adjust the input parameters to ventilator 220 to achieve the desired goals in terms of the targeted PIP. This can also be done by considering changes in the PEEP while also considering \hat{Q}=max|Q(t)| (for any PIP value). One of ordinary skill in the art should appreciate that the adjustment of PEEP values corresponds to values of \hat{Q}. Immediate corrective actions can also be taken, where [symbol] agent 512 adjusts the PEEP that corresponds to \hat{Q} reaching critical levels. One of ordinary skill in the art should appreciate that we are considering the mode of pressure-controlled ventilation (PCV).

Further, in an embodiment, RL_{PV} agent 514 can govern pressure P(t) and volume V(t) associated with ventilator 220 by controlling input parameters to ventilator 220. For example, RL_{PV} agent 514 can dynamically adjust the input parameters to ventilator 220 in terms of the targeted RR. This can be done, for example, by considering changes to the inspiratory time Tᵢₙₛₚ and, consequently, the TCT.

Markov Decision Process for an [symbol] Agent

Consistent with an embodiment, a Markov decision process (MDP) for [symbol] agent 512 can consider the following operating ranges: (a) valid operating ranges in terms of changes in PEEP (with respect to each PIP value, e.g., max|Q(t)|∈[60, 120] at inspiration and expiration); and (b) warning ranges for “critical PEEP values” (with respect to fixed PIP values) (e.g., max|Q(t)|<60 OR max|Q(t)|>120 at inspiration and expiration).

Consistent with an embodiment, the MDP developed can consider incremental variations of PEEP for each constant PIP variation. The operating considered include: PEEP∈[3, 15], PIP∈[20, 40], where IP=PEEP−PIP.

The state space [symbol] for [symbol] agent 512 can include the range of PEEP values, which can correspond to max|Q(t)| at inspiration and expiration, and can be based on the various heights of Q that correspond to variations of PEEP values.

The action space [symbol] for [symbol] agent 512 includes the adjustment of PEEP values: (1) increase PEEP (PEEP=PEEP+1); (2) decrease PEEP (PEEP=PEEP−1); (3) keep PEEP the same (PEEP=PEEP); (4) increase PIP (PIP=PIP+1); (5) decrease PIP (PIP=PIP−1); and (6) keep PIP the same (PIP=PIP).

Consistent with an embodiment, the set of rewards [symbol] for an MDP for [symbol] agent 512 can be based on the following schema: (1) PEEP in operating range: Rₜ=20−|PEEP−PEEP_{baseline}|; (2) PEEP not in operating range: Rₜ=−80; (3) PIP in operating range: Rₜ=20−|PIP−PIP_{baseline}|; (4) PIP not in operating range: Rₜ=−80; (5) PIP=PIP_{tgt}: Rₜ=40; (6) IP in operating range: Rₜ=20; (7) IP not in operating range: Rₜ=−80.

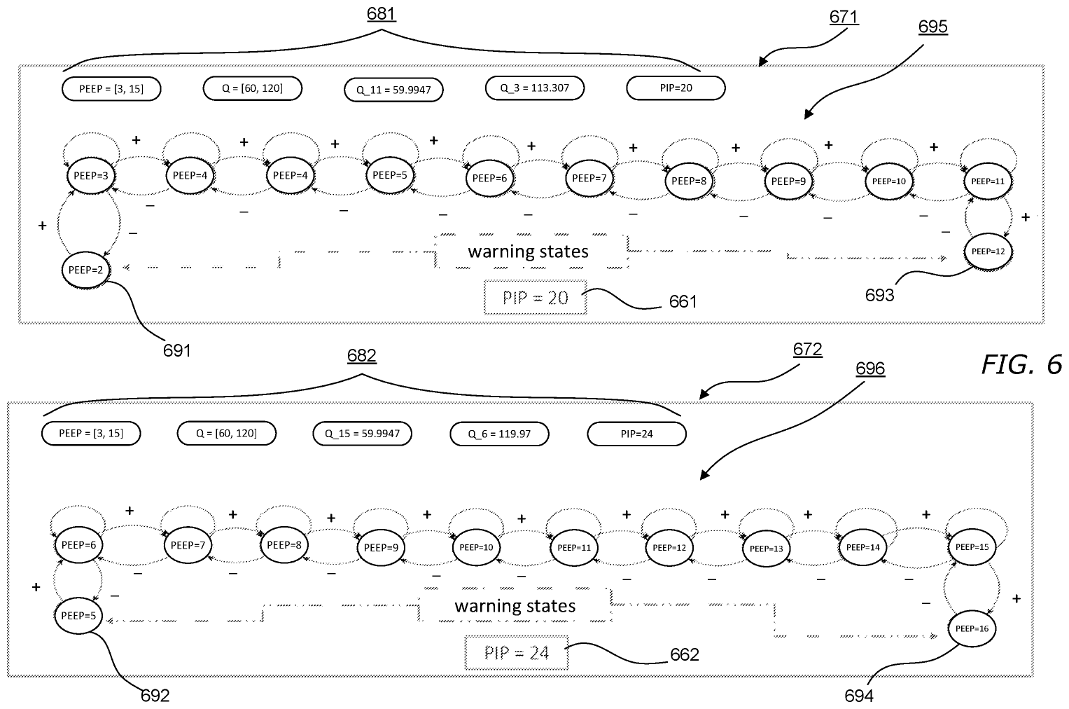

FIG. 6 depicts exemplary MDP transition graphs 671 and 672 for [symbol] agent 512, consistent with the [symbol] , [symbol] , and [symbol] set forth above. Values 695 and 696 depict the range of PEEP values for a fixed PIP value (i.e., PIP values 661 and 662). Values 691 and 693 depict the “warning states” for graph 671, and values 692 and 694 depict the “warning states” for graph 672. Parameters 681 and 682 include a range of PEEP values, a fixed PIP value, and values associated with \hat{Q}=max|Q(t)|, and “warning” states associated with [symbol] (t) values at <60 and >120.

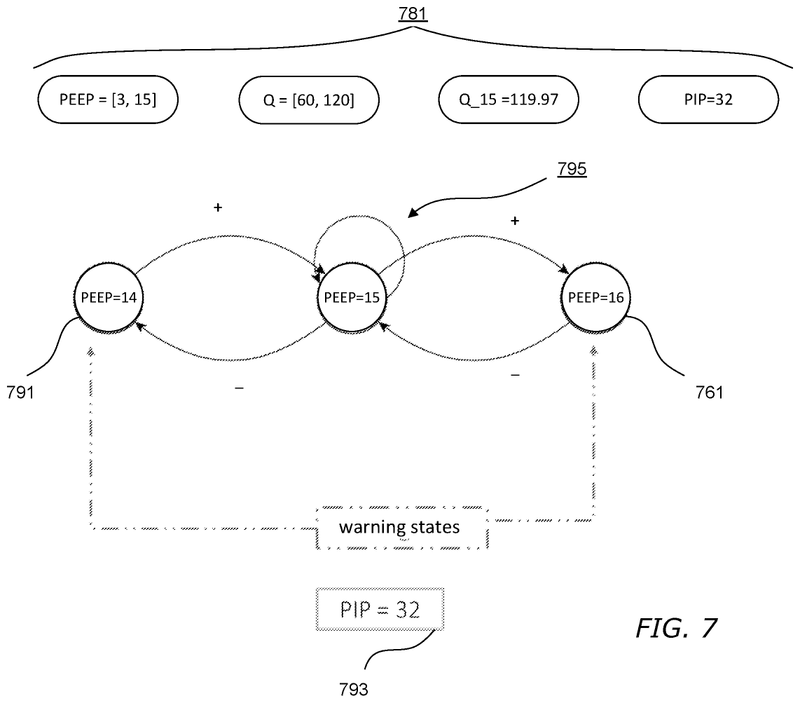

For illustrative purposes only, FIG. 7 depicts an extreme case, where PEEP=15 (value 795) is the only available state not in the “warning state” associated with [symbol] (t) values at <60 and >120 (values 791 and 793). The fixed PIP value (value 761) is PIP=32. One of ordinary skill in the art should appreciate that the transition graph depicted in FIG. 7 is also needed so that [symbol] agent 512 can learn what to do in this scenario.

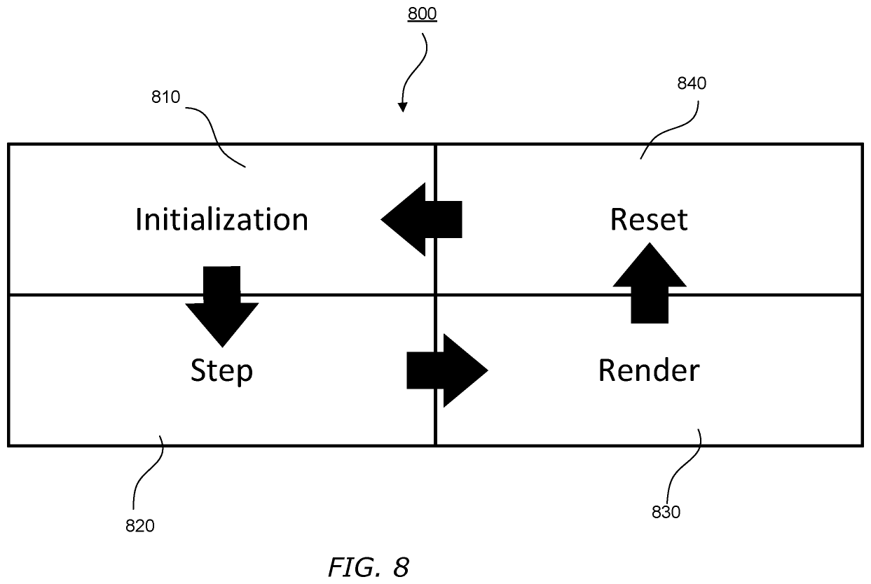

FIG. 8 is a high-level diagram illustrating an Artificial Intelligence (AI) Gym environment 800 for use with aspects of the reinforcement-learning associated with FIG. 5. With respect to [symbol] agent 512, the Initialization component 810 can be characterized by the following: (1) defines the action space A_{Q} as proposed above for the MDP for [symbol] agent 512; (2) defines observation space (also known as the state space) S_{Q}; as discussed above, this can depend on the PEEP, PIP, and IP ranges; and (3) the ventilation length can be initialized (default=60 s).

With respect to [symbol] agent 512, the Step component 820 can be characterized by the following: (1) incrementally changes the PEEP, PIP, and IP; (2) the rewards (as described above [symbol] ) can be applied; (3) check to see if the ventilation timeframe has been completed; and (4) return the specific location (i.e., Sₜ∈S_{Q}, Aₜ∈A_{Q}) of the MDP.

Similarly, with respect to [symbol] agent 512, the Render component 830 can be characterized by the following: (1) training behavior (via the accumulation of the rewards) can be presented; and (2) the actions of [symbol] agent 512 can be recorded and presented.

Further still, with respect to [symbol] agent 512, the Reset component 840 can be characterized by the following: (1) the environment is reset to the initial PEEP, PIP, and IP settings; and (2) the ventilation time length is also reset (default=60 s).

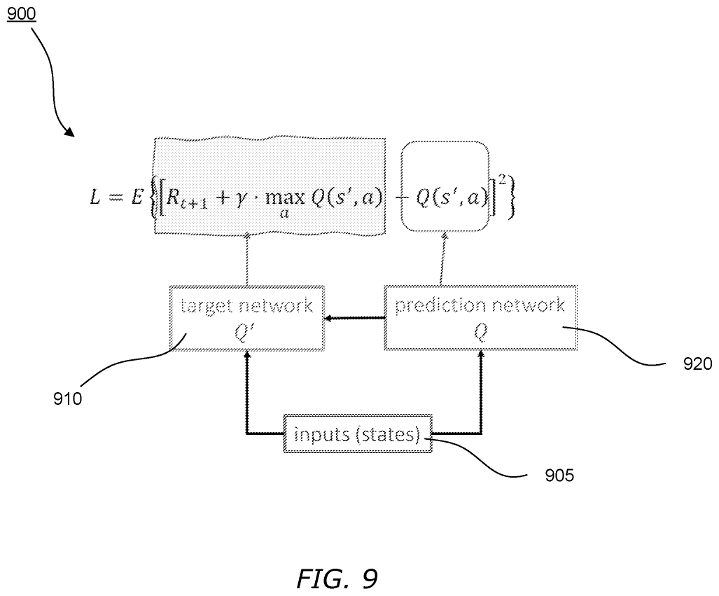

FIG. 9 is a high-level diagram illustration a Deep Q Network formulation for use with aspects of the reinforcement-learning approach of FIG. 5. Specifically, an equation 900 for the Huber loss function/is provided, illustrating the role of inputs 905, target neural network [symbol] ′ 910 and prediction neural network [symbol] 920. In FIG. 9, each neural network (i.e., target neural network [symbol] ′ 910 and prediction neural network [symbol] 920) can be a two-layered rectified linear (ReLU) neural network, where each layer can have 256 nodes. Further, Adam optimization can be used to update the weights of the neural network for faster convergence.

Further still, consistent with an embodiment of the disclosure, Kaiming He initialization can be used to handle the nuances of using ReLU neural networks. The Huber loss function L can be used to mitigate the effects of outliers. Moreover, one of ordinary skill in the art should appreciate that these implementations can make the loss function L more stable, following a consistent downward trend, which can illustrate the consistency of the learning behavior of the [symbol] agent 512.

Consistent with an embodiment of the disclosure, other settings for the Deep Q Network formulation for the [symbol] agent 512 includes: (a) the learning rate used=0.001; (b) epochs=300; (c) updates=every 100 episodes; (d) discount factor γ=0.99; (e) “exploration” and “exploitation” is explored: (e1) the value ε=1 means that the agent is in “exploration”; (e2) the value ε≈0 means that the agent is in “exploitation”. In connection with the latter setting, the value ε=1 was used for [symbol] agent 512 to begin exploration, the value ε was decremented by Δε=5×10⁻⁴ and, and the value ε=0.01 denotes when [symbol] agent 512 is at exploitation.

As used herein, the value of 1 epoch means one training cycle (i.e., forward and backward passes) in the neural network. Typically, training can take more than a few epochs to allow for better generalization. This is heuristically chosen depending on the neural network configuration given. As used herein, the value of 1 episode means one sequence of states (Sₜ∈S_{Q}), actions (Aₜ∈A_{Q}), and rewards (Rₜ∈R_{Q}). As used herein, the term “exploration” means that the agent (i.e., [symbol] agent 512, for example) knows nothing (is a novice) about what needs to be learned. Thus, one of ordinary skill in the art should appreciate that the value ε=1 is associated with random choices. As used herein, the term “exploitation” means that the agent (i.e., [symbol] agent 512, for example) knows much—that the agent is an expert. Thus, one of ordinary skill in the art should appreciate that the value ε≈0 is associated with highly deterministic choices.

Consistent with the AI Gym Environment 800 set forth above, the implementation of the MDP for [symbol] agent 512 can be tested to see if it is implemented robustly. Consistent with an embodiment, a testing strategy can consist of: (1) traversing forward through each state of the MDP for [symbol] agent 512 in the AI Gym environment 800; (2) print out each state and action accordingly (e.g., “State 1=______”, “Action 1=______”, etc.); and (3) traverse backward through each state of the MDP for [symbol] agent 512 in the AI Gym environment 800.

Consistent with an embodiment of the disclosure, testing was done via considering an ideal mechanical ventilator (MV) with lung simulation in MATLAB by M. Jaber et al. (2020) for pressure-controlled ventilation (PCV) mode (implemented in MATLAB Simulink). In addition, the input parameters (RR, I/E, PEEP, PIP, and consequently IP) were incrementally adjusted. Specifically, only one parameter was incrementally adjusted while holding the other parameters constant. The incremental and prominent changes of each MV output curve (i.e., flow output Q (t), pressure output P (t), and volume output V (t)) were recorded.

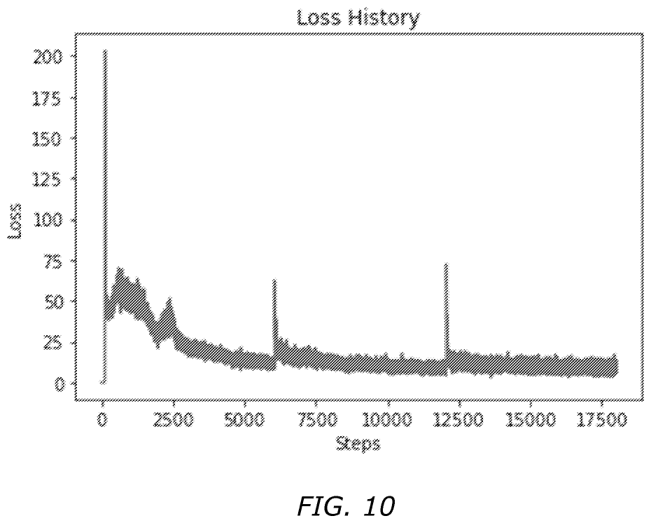

The results of a loss history test for [symbol] agent 512 according to the strategy described above is depicted in FIG. 10. The loss history (i.e., the Huber loss function L) in FIG. 10 is over a period of 60 seconds. Each training step represents an epoch (or update) within the 60 second period. Thus, the total number of steps is 18,000 steps.

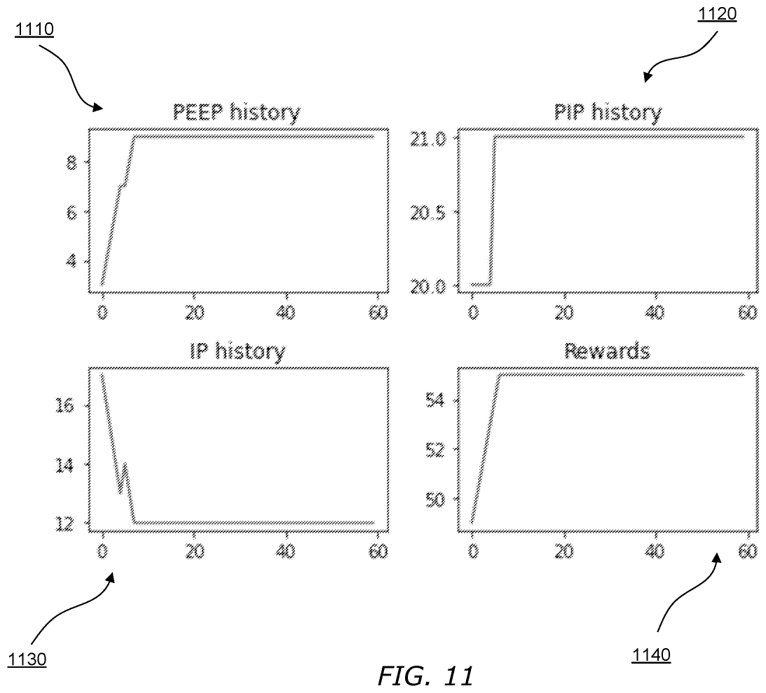

FIG. 11 depicts the actions made by [symbol] agent 512 during the 60 second period and the training history (via the accumulated rewards). Graphs 1110, 1120, and 1130 depict exemplary outputs of the mechanical ventilator (MV) input adjustments with respect to the PEEP, PIP, and IP. Graph 1140 depicts the rewards trend. Graph 1140 illustrates that [symbol] agent 512 is performing well in terms of learning these adjustments.

Table 1 below provide a metrics summary consistent with an embodiment of this disclosure.

Percentage of Achieved Targeted PIP Values for RL_{Q} [Target PIP =
25, Total Number of Occurrences = 273]

| | | Percentage of | Percentage of |
|---|---|---|---|
| Absolute Difference | | Occurrences > | Occurrences < |
| in Achieved PIP | Percentage of | Targeted PIP | Targeted PIP |
| Value | Occurrences | Value | Value |
| 0 | 76.19 | 0 | 0 |
| 1 | 4.76 | 0 | 100 |
| 2 | 4.76 | 0 | 100 |
| 3 | 4.76 | 0 | 100 |
| 4 | 4.76 | 0 | 100 |
| 5 | 4.76 | 0 | 100 |

Markov Decision process for an RL_{PV} agent

Consistent with an embodiment, a Markov decision process (MDP) for RL_{PV} agent 514 can consider the following operating ranges: (a) valid operating ranges in terms of changes in PEEP (with respect to each PIP value); (b) valid operating ranges in terms of changes in the respiratory rate (RR); (c) valid operating ranges in terms of changes in the inspiratory/expiratory (I/E) ratio (where the I/E ratios considered are {0.25, 0.33, 0.5, 0.75}); (d) the range of the total cycle time (TCT); and (e) the following termination conditions: (e1) RL_{PV} agent 514 terminates its adjustments when both the I/E and RR targets are met within the 60 second period; and (e2) RL_{PV} agent 514 terminates the process when the 60 second period is reached.

In terms of the TCT, the following equations are considered: (1) TCT=Tᵢₙₛₚ+Tₑₓₚ; and (2) RR=(1 min)/TCT.

In addition, the following operating ranges are considered: PEEP∈[3, 15], PIP∈[20, 40] {NOTE: IP=PEEP−PIP}, RR∈[12, 20]; and the I/E ratios presented above are considered (i.e., {0.25, 0.33, 0.5, 0.75}).

The state space S_{PV} for RL_{PV} agent 514 can be based on the total cycle time (TCT) based on Tᵢₙₛₚ and Tₑₓₚ. This state space considers changes in the RR and I/E ratios for a fixed combination of PEEP and PIP values.

The action space A_{PV} can include the adjustment of the I/E and RR. For example: (1) increase I/E (I/E=I/E+1); (2) decrease I/E (I/E=I/E−1); (3) keep IE the same (I/E=I/E); (4) increase RR (RR=RR+1); (5) decrease RR (RR=RR−1); (6) keep RR the same (RR=RR).

Consistent with an embodiment, the set of rewards R_{PV} for an MDP for RL_{PV} agent 514 can be based on the following schema: (a) RR in operating range: Rₜ=3×(20−|RR−RR_{baseline}|); (b) RR not in operating range: Rₜ=−1000; (c) I/E in operating range: Rₜ=30−|I/E−I/E_{baseline}| (d) I/E not in operating range: Rₜ=−1000; (e) RR=RR_{tgt}: Rₜ=90; (f) I/E=I/E_{tgt}: Rₜ=60; (g) RR=RR_{tgt} AND I/E=1/E_{tgt}: Rₜ=1000 (where “AND” is the logical AND operation, which means that both conditions must be met to receive a reward of 1000).

based on the following schema: (1) PEEP in operating range: Rₜ=20−|PEEP−PEEP_{baseline}|; (2) PEEP not in operating range: Rₜ=−80; (3) PIP in operating range: Rₜ=20−|PIP−PIP_{baseline}|; (4) PIP not in operating range: Rₜ=−80; (5) PIP=PIP_{tgt}: Rₜ=40; (6) IP in operating range: Rₜ=20; (7) IP not in operating range: Rₜ=−80.

As discussed above, FIG. 8 is a high-level diagram illustrating an Artificial Intelligence (AI) Gym environment 800 for use with aspects of the reinforcement-learning associated with FIG. 5. With respect to RL_{PV} agent 514, the Initialization component 810 can be characterized by the following: (1) defines the action space A_{PV} as proposed in the MDP for RL_{PV} agent 514; (2) defines observation space (also known as the state space) S_{PV} for RL_{PV} agent 514; as discussed above, this can depend on the I/E and RR ranges; (3) the ventilation length is initialized (default=60 s).

With respect to RL_{PV} agent 514, the Step component 820 can be characterized by the following: (1) incrementally change the I/E and RR; (2) rewards (as discussed above R_{PV}) are applied.; (3) checks to see if the ventilation timeframe has been completed; and (4) returns the specific location (i.e., Sₜ∈S_{PV}, Aₜ∈A_{PV}) of the MDP for RL_{PV} agent 514.

Similarly, with respect to RL_{PV} agent 514, the Render component 830 can be characterized by the following: (1) training behavior (via the accumulation of the rewards) is presented; and (2) the actions of RL_{PV} agent 514 are recorded and presented

Further still, with respect to RL_{PV} agent 514, the Reset component 840 can be characterized by the following: (1) the environment is reset to the initial PEEP, PIP, and IP settings; and (2) the ventilation time length is also reset (default=60 s).

Again, as discussed above, FIG. 9 is a high-level diagram illustration a Deep Q Network formulation for use with aspects of the reinforcement-learning approach of FIG. 5. Specifically, an equation 900 for the Huber loss function L is provided, illustrating the role of inputs 905, target neural network [symbol] ′ 910 and prediction neural network [symbol] 920. In FIG. 9, as discussed above, each neural network (i.e., target neural network [symbol] ′ 910 and prediction neural network [symbol] 920) can be a two-layered rectified linear (ReLU) neural network, where each layer can have 256 nodes. Further, Adam optimization can be used to update the weights of the neural network for faster convergence.

Further still, consistent with an embodiment of the disclosure, Kaiming He initialization can be used to handle the nuances of using ReLU neural networks. The Huber loss function L can be used to mitigate the effects of outliers. Moreover, one of ordinary skill in the art should appreciate that these implementations can make the loss function L more stable, following a consistent downward trend, which can illustrate the consistency of the learning behavior of the RL_{PV} agent 514.

Consistent with an embodiment of the disclosure, other settings for the Deep Q Network formulation for the RL_{PV} agent 514 includes: (a) the learning rate used=0.001; (b) epochs=300; (c) updates=every 50 episodes; (d) discount factor γ=0.99; (e) “exploration” and “exploitation” is explored: (e1) the value E=1 means that the agent is in “exploration”; (e2) the value ε≈0 means that the agent is in “exploitation”. In connection with the latter setting, the value ε=1 was used for RL_{PV} agent 514 to begin exploration, the value E was decremented by Δε=5×10⁻⁴ and, and the value ε=0.01 denotes when RL_{PV} agent 514 is at exploitation.

Further as discussed above, the value of 1 epoch means one training cycle (i.e., forward and backward passes) in the neural network. Typically, training can take more than a few epochs to allow for better generalization. This is heuristically chosen depending on the neural network configuration given. As used herein, the value of 1 episode means one sequence of states (Sₜ∈S_{PV}), actions (Aₜ∈A_{PV}), and rewards (Rₜ∈R_{PV}). As used herein, the term “exploration” means that the agent (i.e., RL_{PV} agent 514, for example) knows nothing (is a novice) about what needs to be learned. Thus, one of ordinary skill in the art should appreciate that the value ε=1 is associated with random choices. As used herein, the term “exploitation” means that the agent (i.e., RL_{PV} agent 512, for example) knows much—that the agent is an expert. Thus, one of ordinary skill in the art should appreciate that the value ε≈0 is associated with highly deterministic choices.

Consistent with the AI Gym Environment 800 set forth above, the implementation of the MDP for RL_{PV} agent 514 can be tested to see if it is implemented robustly. Consistent with an embodiment, a testing strategy can consist of: (1) traversing forward through each state of the MDP for RL_{PV} agent 514 in the AI Gym environment 800 as described above; (2) print out each state and action accordingly (e.g., “State 1=______”, “Action 1=______”, etc.); and (3) traverse backward through each state of the MDP for RL_{PV} agent 514 in the AI Gym environment 800.

Consistent with an embodiment of the disclosure, testing was done via considering an ideal mechanical ventilator (MV) with lung simulation in MATLAB by M. Jaber et al. (2020) for pressure-controlled ventilation (PCV) mode (implemented in MATLAB Simulink). In addition, the input parameters (RR, I/E, PEEP, PIP, and consequently IP) were incrementally adjusted. Specifically, only one parameter was incrementally adjusted while holding the other parameters constant. The incremental and prominent changes of each MV output curve (i.e., flow output Q (t), pressure output P (t), and volume output V (t)) were recorded. For RL_{PV} agent 514, This also includes examining changes in the total cycle time (TCT), which includes the RR, the I/E ratio, the inspiratory time Tᵢₙₛₚ, and the expiratory time Tₑₓₚ.

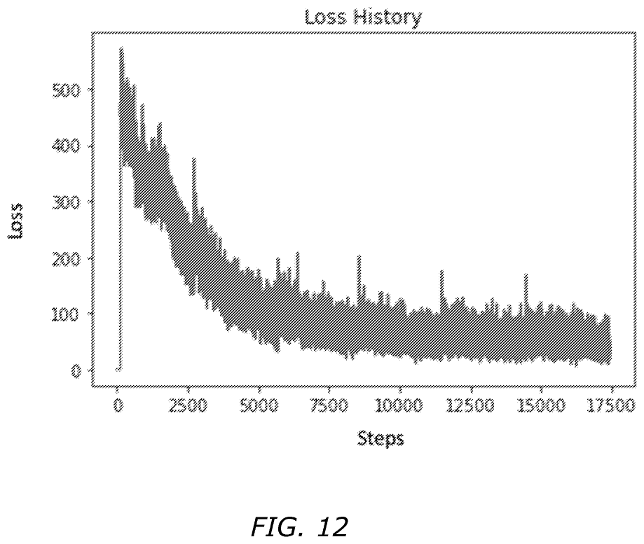

The results of a loss history test for RL_{PV} agent 514 according to the strategy described above is depicted in FIG. 12. The loss history (i.e., the Huber loss function L) in FIG. 12 is over a period of 60 seconds. Each training step represents an epoch (or update) within the 60 second period. Thus, the total number of steps is 18,000 steps.

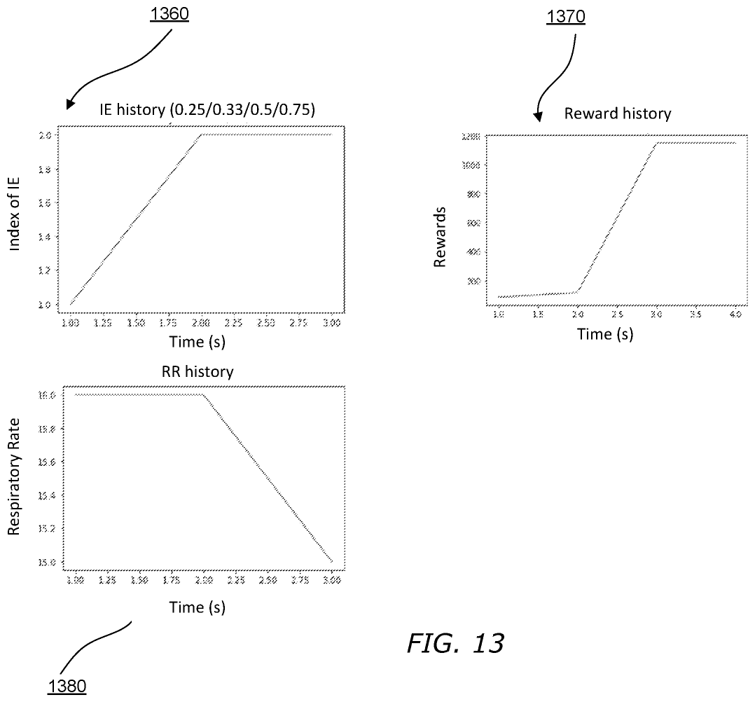

FIG. 13 depicts the output of the mechanical ventilator (MV) input with respect to the RR and I/E (graphs 1380 and 1360, respectively) and the training history (via the accumulated rewards, i.e., graph 1370).

Table 2 below provide an RR metrics summary consistent with an embodiment of this disclosure.

Percentage of Achieved Targeted RR Values for RL_{PV} [Target RR =
15, Total Number of Occurrences = 36]

| | | Percentage of | Percentage of |
|---|---|---|---|
| Absolute Difference | | Occurrences > | Occurrences < |
| in Achieved RR | Percentage of | Targeted RR | Targeted RR |
| Value | Occurrences | Value | Value |
| 0 | 66.67 | 0 | 0 |
| 1 | 11.11 | 0 | 100 |
| 2 | 11.11 | 0 | 100 |
| 3 | 11.11 | 0 | 100 |

Table 3 below provide an I/E metrics summary consistent with an embodiment of this disclosure.

Percentage of Achieved Targeted I/E Values for RL_PV [Target IE =
0.5 (index = 2), Total Number of Occurrences = 36]

| Absolute | | Percentage of | |
|---|---|---|---|
| Difference in | | Occurrences > | Percentage of |
| Achieved PIP | Percentage of | Targeted IE | Occurrences < |
| Value | Occurrences | Value | Targeted IE Value |
| 0 | 83.33 | 0 | 0 |
| 1 | 16.67 | 0 | 100 |

### Rules-Based Development for an RL_{B} Agent

Consistent with an embodiment of the disclosure, a rules-based development for RL_{B} agent 510 can include the following: (1) orchestrating decisions regarding the initial decision in terms of managing the partial pressure of carbon dioxide in arterial blood (PaCO2); (2) orchestrating decisions regarding managing the tidal volume; (3) orchestrating decisions regarding managing the respiratory rate (RR); and (4) automatically reporting metrics and alarms to clinicians to make interactive measures with the ventilator 220. These can include (but are not limited to): (a) noting and treating patient asynchronous behavior; (b) decreasing fever to decrease carbon dioxide (CO2) production.; and (c) reporting time-dependent adjustments for ventilator 220 on a periodic basis.

Systems consistent with the current embodiment can be adapted to patients in real-time in clinical environments, paired with trust monitors to oversee the assuredness of the system and raise flags if human intervention is required. One of ordinary skill in the art would appreciate that systems consistent with the current embodiment can be applied to autonomous system in other patient-care environments, such as autonomous systems in an ICU environment.

Systems consistent with the current embodiment can recognize patterns that require escalation or de-escalation of ventilator support. Further still, systems consistent with the current disclosure can recognize divergence between expected and actual behaviors that signify events requiring clinician intervention, such as accidental extubation, circuit disconnects, or device malfunction.

Consistent with the current disclosure, a digital twin of the patient-ventilator dyad can be structured to prioritize a state-space that (1) is identifiable from the limited data available, (2) provides actionable information for adjusting ventilatory settings, and (3) can predict responses to changes. Consistent with this disclosure, the digital twin can be continuously adjusted to conform to an individual patient (either real or in silico). Small divergences between the digital twin and patient are necessary by-products of in-complete information availability and simplifications made to enable identifiability. However large divergences and/or impossible states will signal leaving the model's validity zone, requiring clinician help.

The digital twin state can be further configured to function in a set of representative scenarios that include (1) common patient pathophysiology (such as V/Q mismatch and dead space from ARDS, diffusion limitation from pulmonary edema, shunt from pneumothorax), (2) common ventilator/circuit faults (e.g., endotracheal tube migration/displacement, circuit leaks, deliberate patient disconnection for treatment, etc.), and (3) sensor faults (e.g., faulty pulse oximetry or airway flow).

Each of the above-mentioned scenarios can be simulated in the digital twin environment, and the outcomes can be analyzed using data error detection, comparison with predicted results, and validation by clinical experts to ensure accuracy. Statistical reliability analysis can be performed to investigate temporal changes with respect to the ventilator inputs, such as tidal volume, positive end-expiratory pressure (PEEP), respiratory rate, and inspiratory airflow, and the ventilator visual outputs, like pressure, flow, and volume that are based on each patient's subject-specific response. One of ordinary skill in the art would appreciate that a benefit to this analysis is any results can be used to develop automatic control policies for ventilator management, which are described below.

Because a digital twin consistent with the current disclosure can be used to detect significant divergence between models, an element of the assurance subsystem for autonomous ventilator control can be a continuous checking of observed changes in the patient's physiological measures against the predictions of the computational model. If these changes differ significantly from expectations, then the system can automatically sound an alert and request intervention from a human clinician. Trust monitor algorithms consistent with the current disclosure can be used to analyze the data continuously to detect anomalies or red flags, e.g., asynchronous breathing, and secondly, continuously check between the computational models' predictions and the observed data. Under such control, trust monitors consistent with the current disclosure can enable one to later define a set of safety boundaries for autonomous ventilators. Consistent with the current disclosure, time alignment of the various signals and building algorithms to reset the alignment periodically or whenever a misalignment is suspected can be performed. For example, the system can operate algorithms to recognize breaths when a patient's breathing is not synced with the ventilator.

As mentioned above, one of ordinary skill in the art would appreciate that an application of the digital twin system and analysis described herein can discover control policies for assured autonomous ventilators in ICU.

The foregoing descriptions have been presented for purposes of illustration. They are not exhaustive and are not limited to precise forms or embodiments disclosed. Modifications and adaptations of the embodiments will be apparent from consideration of the specification and Exhibits and practice of the disclosed embodiments.

Moreover, while illustrative embodiments have been described herein, and in the Exhibits, the scope includes any and all embodiments having equivalent elements, modifications, omissions, combinations (e.g., of aspects across various embodiments), adaptations and/or alterations based on the present disclosure. The elements in the claims are to be interpreted broadly based on the language employed in the claims and not limited to examples described in the present specification or during the prosecution of the application, which examples are to be construed as nonexclusive.

Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

Other embodiments will be apparent from consideration of the specification and Exhibits and practice of the embodiments disclosed herein. It is intended that the specification, Exhibits, and examples be considered as example only, with a true scope and spirit of the disclosed embodiments being indicated by the following claims.

1. A system for generating an assurance value for autonomous ventilation of a patient, said system comprising:
at least one computer;
at least one storage device;
a ventilator coupled to said at least one computer, said ventilator associated with the patient; and
at least one device for acquiring patient physiological data associated with the patient, said at least one computer coupled to said at least one device for receiving acquired patient physiological data;
said at least one storage device storing instructions that, when executed by the at least one computer, cause the at least one computer to perform a method of generating the assurance value, said method comprising:
determining at least one adaptive patient digital twin and a correlated ventilator model;
receiving said acquired patient physiological data during a first time period;
processing the at least one adaptive patient digital twin and the correlated ventilator model to generate expected patient physiological data during a second time period after the first time period;
receiving said acquired patient physiological data during the second time period;
processing the acquired patient physiological data during the second time period and the expected patient physiological data during the second time period to generate an assurance value associated with the at least one adaptive patient digital twin and a correlated ventilator model.

2. The system for generating an assurance value of claim 1, wherein said ventilator is configured to provide first ventilator data corresponding to said first time period to said computer;
wherein said processing the at least one adaptive patient digital twin and the correlated ventilator model further includes processing said first ventilator data to generate expected ventilator data during said second time period;
wherein receiving said acquired patient physiological data during the second time period includes receiving second ventilator data corresponding to said second time period; and
wherein processing the acquired patient physiological data during the second time period and the expected patient physiological data during the second time period includes processing the second ventilator data received during the second time period and the expected ventilator data during the second time period.

3. The system for generating an assurance value of claim 1, wherein said assurance value is generated based upon a threshold.

4. The system for generating an assurance value of claim 1, wherein divergence between said acquired patient physiological data during the second time period and the expected patient physiological data during the second time period is used to generate a modification of said at least one adaptive patient digital twin and correlated ventilator model.

5. The system for generating an assurance value of claim 1, wherein said method of generating the assurance value further comprises:
generating a model for patient prognosis during a third time period that provides an indication of patient prognosis at a fixed time past the third time period.

6. The system for generating an assurance value of claim 5, wherein said method of generating the assurance value further comprises:
providing an alert when said indication of patient prognosis at the fixed time past the third time period is below a set value.

7. The system for generating an assurance value of claim 1, wherein said at least one adaptive patient digital twin and correlated ventilator model is configured to model a patient pathophysiology.

8. The system for generating an assurance value of claim 7, wherein said patient pathophysiology is at least one of: a V/Q mismatch; a dead space from ARDS; a diffusion limitation from pulmonary edema; and a shunt from pneumothorax.

9. The system for generating an assurance value of claim 1, wherein said at least one adaptive patient digital twin and correlated ventilator model is configured to model at least one of: a ventilator fault and a circuit fault.

10. The system for generating an assurance value of claim 9, wherein said ventilator fault and said circuit fault is at least one of: an endotracheal tube migration; an endotracheal tube displacement; a circuit leak; and a patient disconnection for treatment.

11. The system for generating an assurance value of claim 1, wherein said at least one adaptive patient digital twin and correlated ventilator model is configured to model a sensor fault.

12. The system for generating an assurance value of claim 1, wherein said sensor fault is at least one of: a pulse oximetry sensor fault and an airway flow sensor fault.

13. A method of generating an assurance value for autonomous ventilation of a patient by a ventilator, the method comprising:
determining at least one adaptive patient digital twin and a correlated ventilator model;
acquiring physiological data associated with the patient during a first time period, the acquired physiological data being acquired by a device;
processing the at least one adaptive patient digital twin and the correlated ventilator model to generate expected patient physiological data during a second time period after the first time period;
receiving said acquired physiological data during the second time period;
processing the acquired physiological data during the second time period and the expected patient physiological data during the second time period to generate an assurance value associated with the at least one adaptive patient digital twin and a correlated ventilator model.

14. The method of claim 13, wherein said ventilator is configured to generate first ventilator data corresponding to said first time period;
wherein said processing the at least one adaptive patient digital twin and the correlated ventilator model further includes processing said first ventilator data to generate expected ventilator data during said second time period;
wherein receiving said acquired physiological data during the second time period includes receiving second ventilator data corresponding to said second time period; and
wherein processing the acquired patient physiological data during the second time period and the expected patient physiological data during the second time period includes processing the second ventilator data received during the second time period and the expected ventilator data during the second time period.

15. The method of generating an assurance value of claim 13, wherein said assurance value is generated based upon a threshold.

16. The method of generating an assurance value of claim 13, wherein divergence between said acquired physiological data during the second time period and the expected patient physiological data during the second time period is used to generate a modification of said at least one adaptive patient digital twin and correlated ventilator model.

17. The method of generating an assurance value of claim 13 further comprising:
generating a model for patient prognosis during a third time period that provides an indication of patient prognosis at a fixed time past the third time period.

18. The method of generating an assurance value of claim 17 further comprising:
providing an alert when said indication of patient prognosis at the fixed time past the third time period is below a set value.

19. The method of generating an assurance value of claim 13, wherein said at least one adaptive patient digital twin and correlated ventilator model is configured to model a patient pathophysiology.

20. The method of generating an assurance value of claim 13, wherein said at least one adaptive patient digital twin and correlated ventilator model is configured to model at least one of: a ventilator fault; a circuit fault; and a sensor fault.
